(19) United States
(12) Patent Application Publication
(10) Pub. No.: US 2014/0256597 A1
(43) Pub. Date: Sep. 11, 2014

(54) Method of amplifying and labeling the mRNA sample for mRNA microarray

(71) Applicant: Weigong He, Sugar Land, TX (US)

(72) Inventor: Weigong He, Sugar Land, TX (US)

(73) Assignee: APPLIED HYBRIDIZATION BIOLAB, Sugar Land, TX (US)

(21) Appl. No.: 13/794,725

(22) Filed: Mar. 11, 2013

(51) Int. Cl. C12Q 1/68

(52) U.S. Cl. CPC C12Q 1/6806
USPC 506/26

(57) ABSTRACT

Disclosed is method of amplifying and labeling mRNA sample for the mRNA microarray. The invention utilizes the specific synthesized single strand oligonucleotide (ssDNA) poly-T to hybridize the complementary poly-A in the tail of the mRNA; converts the hybridized mRNA to the cDNA; hybridizes the mRNA detection probe on the mRNA microarray chip with the cDNA; amplifies the hybridized cDNA through extending the polymer based on the ssDNA; labels the amplified cDNA by integrating the fluorescent modified nucleotide into the amplified duplex oligonucleotide during polymer extension; verifies the amplified labeled cDNA through detecting the fluorescent signal. The fluorescent signal from the mRNA detection probe spot on the mRNA microarray chip will indicate the presence of the detected mRNA and the quantity of the fluorescent signal from the mRNA detection probe sport will be directly proportional to the amount of the fluorescent modified nucleotide in the amplified duplex oligonucleotide.

### RELATED U.S. APPLICATION DATA

Provisional application No. 61/685,194, filed on Mar. 14, 2012.

### FIELD OF THE INVENTION

The present invention relates generally to the field of detecting nucleic acid and specifically to amplifying and labeling the message RNA (mRNA) sample for the mRNA microarray.

The concepts and methods of the present invention are applicable in any animals, microbes and plants.

### BACKGROUND OF THE INVENTION

The mRNA microarray is a commonly used molecular biological technique for studying the multiple mRNA expression. With a single hybridization, the level of hundreds or even thousands mRNA expression can be estimated for the sample of cell or tissue.

At present the mRNA microarray technique is involved five portions: making the mRNA microarray chip; amplifying and labeling the mRNA sample with the fluorophore; hybridizing the labeled mRNA sample with the mRNA detecting probe on the mRNA microarray chip; scanning the hybridized mRNA microarray chip; and analyzing the hybridized pattern.

Although the mRNA microarray can detect multiple mRNA expression with a single hybridization but the current mRNA microarray technique has some critical shortcomings including the poor specificity and the low sensitivity comparing with the traditional mRNA detecting techniques such as Northern blot and quantitative real time polymerase chain reaction (qRT-PCR), and sometimes the results from the mRNA microarray cannot match with the data from the Northern blot or the qRT-PCR or the DNA microarray or the protein microarray.

The linear amplification or the exponential amplification or the combination of both amplifications have been using for amplifying and labeling the mRNA sample for the mRNA microarray, however, none of these current mRNA amplifying and labeling methods can get satisfied result in the specificity, sensitivity and reproducibility for the mRNA microarray.

To overcome the shortcomings, the present invention provides a new method to amplify and label the mRNA sample for the mRNA microarray.

### SUMMARY OF THE INVENTION

The invention is based on the concept of molecular biology and the experiment discovery that the nucleic acid fragment can hybridize the complementary nucleic acid under the normal conditions.

The invention is also based on the concept of the polymerization and the primer extension.

The invention involves the following steps: extracting the mRNA from the cell or tissue; synthesizing the specific single strand DNA oligonucleotide (ssDNA); hybridizing the ssDNA poly-deoxythymine (poly-T) with the complementary poly-adenosine (poly-A) in the tail of the mRNA; converting the hybridized mRNA to the complementary DNA (cDNA) through extending the polymer from the 3′end of the hybridized ssDNA poly-T based on the hybridized mRNA; digesting the mRNA from the polymer of cDNA/mRNA with the RNase; hybridizing the cDNA with the mRNA detection probe on the mRNA microarray chip; amplifying the hybridized cDNA through extending the polymer from the 3′end of the hybridized mRNA detection probe based on the ssDNA; labeling the amplified cDNA by integrating the fluorescent modified oligonucleotide into the amplified duplex oligonucleotide during the polymer extension; verifying the amplified labeled cDNA by scanning the hybridized, amplified and labeled mRNA microarray chip; analyzing the hybridized patterns and amplified quantification.

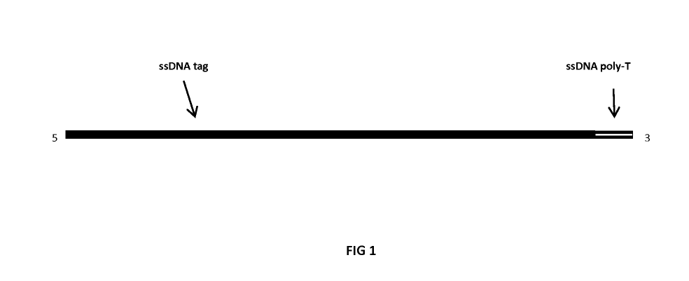

In the invention the ssDNA is composed of two portions: namely, the ssDNA poly-T and the ssDNA tag. At the 3′end, the ssDNA poly-T is poly-deoxythymine oligonucleotide, while at the 5′end the universal nucleic acid sequence oligonucleotide functions as the ssDNA tag as shown in the FIG. 1.

The ssDNA poly-T can hybridize the complementary poly-A in the tail of the detecting mRNA. Working as the primer the hybridized ssDNA poly-T can convert the detected mRNA to the cDNA through extending the polymer from the 3′end of the hybridized ssDNA poly-T based on the hybridized mRNA.

The ssDNA tag cannot contain any complementary nucleic acid sequence of the detecting mRNA, and functions as the tag to amplify the signal of the hybridized detected mRNA.

After converting the detecting mRNA to the cDNA the mRNA is digested from the polymer of the mRNA/cDNA by the RNase.

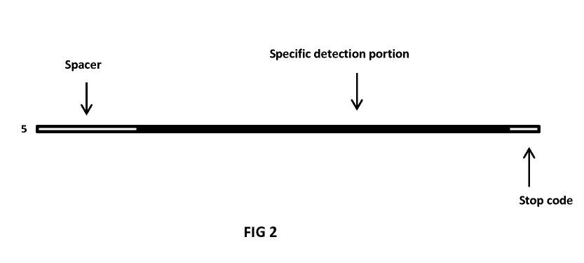

In the invention the mRNA detection probe on the microarray chip is a specific synthesized single strand nucleic acid oligonucleotide and composed of two portions, namely the specific detection portion at the 3′end and the spacer at the 5′end as shown in the FIG. 2.

The specific detection portion of the mRNA detection probe contains the sense sequence nucleic acid oligonucleotide from the 3′end of the detecting mRNA with the sense sequence of the stop code at the 3′terminal.

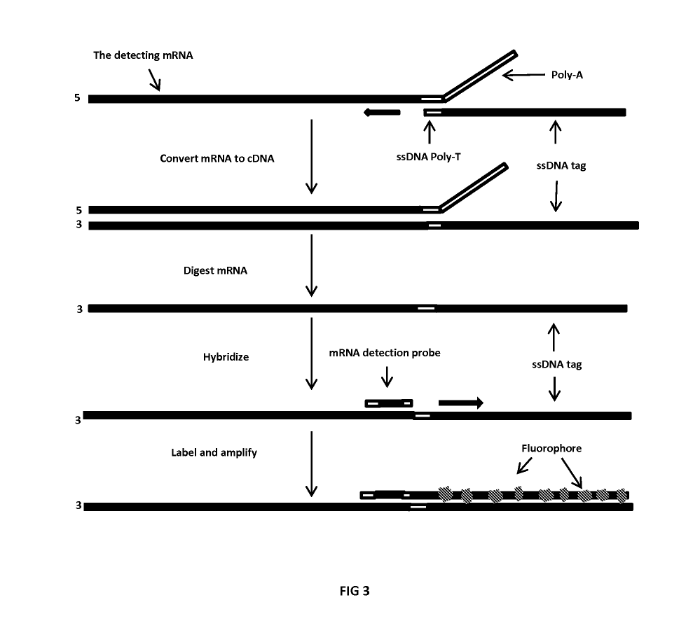

The specific detection portion at the 3′end of the mRNA detection probe on the mRNA microarray chip can hybridize the cDNA of the detecting mRNA. The hybridized cDNA can be amplified through extending the polymer from the 3′end of the hybridized mRNA detection probe based on the ssDNA. The amplified cDNA can be labeled through integrating the fluorescent modified nucleotide into the amplified duplex oligonucleotide during the polymer extension as shown in the FIG. 3.

The spacer of the mRNA detection probe cannot contain any complementary nucleic acid sequence of the detecting mRNA.

The 5′ terminal of the spacer of the mRNA detection probe will be covalently attached to the surface of the mRNA microarray chip.

The spacer of the mRNA detection probe functions as the space to separate the specific detection of the mRNA detection probe with the surface of the mRNA microarray chip to avoid interference from the microarray chip to the hybridization reaction between the cDNA with the specific detection portion of the mRNA detection probe.

The fluorescent signal from the fluorescent modified nucleotide of the amplified duplex oligonucleotide on the mRNA detection probe on the mRNA microarray chip will indicate the presence of the detecting mRNA, and the fluorescent score from the mRNA detection probe spots will be directly proportional to the amount of the fluorescent modified nucleotide in the amplified duplex oligonucleotide, therefore, has close quantitative relationship with the quantity of the detected mRNA.

### BRIEF DESCRIPTIONS OF THE INVENTION

FIG. 1 shows a schematic drawing of the specific synthesized single strand oligonucleotide (ssDNA).

The ssDNA is composed of two portions, namely the ssDNA tag and the ssDNA poly-T.

At the 5′end, the ssDNA tag contains the universal nucleic acid sequence single strand oligonucleotide and works as a tag to amplify the signal of the hybridized and amplified detected mRNA.

The poly-deoxythymine (poly-T) is localized at the 3′end of the ssDNA. The poly-T of the ssDNA can hybridize the complementary poly-A of the mRNA and works as the primer to extend the polymer to convert the mRNA to the cDNA.

FIG. 2 shows a schematic drawing of the mRNA detection probe.

The mRNA detection probe is composed of two portions, namely, the spacer and the specific detection portion.

The spacer is located at the 5′end of the mRNA detection probe. The spacer will function as the space to avoid the obstruction to the hybridization reaction between the cDNA and the mRNA detection probe from the microarray chip surface.

At 3′ end of the mRNA detection probe the specific detection portion contains the same sense sequence from the 3′end of the detecting mRNA with the sense sequence of the stop code at 3′terminal of the specific detection portion of the mRNA detection probe.

The specific detection portion of the mRNA detection probe can hybridize the cDNA of 3′end of the detecting mRNA. The stop code of the 3′terminal of the specific detection portion will guarantee that the polymer only can be extended from the stop code of the detecting mRNA based on the ssDNA.

FIG. 3 shows a schematic drawing of whole process of amplifying and labeling the mRNA sample for the mRNA microarray.

The ssDNA poly-T can hybridize the complementary poly-A in the tail of the detecting mRNA.

The hybridized mRNA can be converted to the cDNA through extending the polymer from the 3′end of the hybridized ssDNA poly-T based on the hybridized mRNA.

The mRNA is digested from the polymer of the mRNA/cDNA by the RNase.

The specific detection portion of the mRNA detection probe on the mRNA microarray chip can hybridize the cDNA of the detecting mRNA.

The hybridized cDNA can be amplified through extending the polymer from the 3′end of the hybridized mRNA detection probe on the mRNA microarray chip based on the ssDNA. The amplified cDNA can be labeled by integrating the fluorescent modified nucleotide into the amplified duplex oligonucleotide during the polymer extension.

### DETAIL DESCRIPTIONS OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials and methods are illustrative only and not intended to be limiting.

The method of amplifying and labeling mRNA sample for the mRNA microarray in the invention herein involves the following steps:

1. Extracting the mRNA from the Cell or Tissue

The mRNA or total RNA can be extracted from the cell or the tissue by using the commercial mRNA extracting kit such as Dynabeads® mRNA Direct Kits (Life technologies, USA) following the instructions.

2. Synthesizing the ssDNA

The ssDNA is a specific synthesized single strand nucleic acid oligonucleotide.

The ssDNA can be synthesized commercially (Integrated DNA technologies (IDT), USA).

The ssDNA is composed of two portions, namely the ssDNA poly-T and the ssDNA tag. At the 3′end, the ssDNA poly-T is poly-deoxythymine oligonucleotide, while at the 5′end the universal nucleic acid sequence oligonucleotide functions as the ssDNA tag as shown in the FIG. 1.

The whole length of the ssDNA used in the invention can be 50-200 nucleotides or longer.

The length of the ssDNA poly-T can be 6-30 deoxythymine.

The ssDNA tag cannot contain any complementary nucleic acid sequence of the detecting mRNA.

The ssDNA can be DNA or RNA or nucleic acid analog.

3. Synthesizing the mRNA Detection Probe

The mRNA detection probe is a specific synthesized single strand nucleic acid oligonucleotide.

The mRNA detection probe can be commercially synthesized (IDT, USA).

The mRNA detection probe on the microarray chip is composed of two portions, namely the specific detection portion at the 3′end and the spacer at the 5′end as shown in the FIG. 2.

The length of the specific detection portion in the mRNA detection probe can be 30-100 nucleotides or longer.

The nucleic acid sequence of the specific detection portion is same with nucleic acid sense sequence at the 3′end of the detecting mRNA.

The sequence of the last 3 nucleic acid at 3′end of the specific detection portion is the sense stop code sequence of the detecting mRNA.

The spacer in the mRNA detecting probe cannot contain any complementary nucleic acid sequence of the detecting mRNA.

The length of the spacer can be 8-10 nucleotides.

The spacer of the mRNA detection probe function as a space to separate the microarray chip surface with the specific detection portion of the mRNA detection probe to avoid the obstruction to the hybridization reaction.

The mRNA detection probe can be DNA or RNA or nucleic acid analog.

4. Making the Detecting mRNA Microarray Chip

The detecting mRNA microarray chip can be ordered commercially (LS science, USA).

The mRNA detecting probe is used for making the detecting mRNA microarray chip in the invention.

The 5′terminal of the spacer of the mRNA detection probe is covalently attached to the surface of the mRNA microarray chip.

5. Converting the mRNA to the cDNA

The ssDNA poly-T can hybridize the complementary poly-A of the tail of the mRNA. The hybridized mRNA can be converted to the cDNA through extending the polymer from the 3′end of the hybridized ssDNA poly-T based on the hybridized mRNA under action of the reverse transcriptase as shown in the FIG. 3.

The mRNA can be transferred to the cDNA in the following conditions: adding 1.0 μg total RNA or 10 ng mRNA, 1.0 μl 30 pM ssDNA, incubating at 70° C. for 2 minutes, then quickly chill on ice, adding 2.0 μl 1 mM dNTPs mix, 2.0 μl 10× reverse transcriptase reaction buffer, 5 units reverse transcriptase (New England Biolab (NEB), USA), DEPC treated sterile distill water to the final volume of 20.0 μl. Incubating solution at 42° C. for 60 minutes.

6. Digesting the mRNA from the Polymer of the cDNA/mRNA

After converting the mRNA to the cDNA, the RNase I/H are added into the reaction solution to digest the mRNA from the cDNA/mRNA polymer as shown in the FIG. 3.

The 5 unites RNase If/H (NEB, USA) are added into the reaction solution.

Incubating at 37° C. for 30 minutes.

Purifying the cDNA solution through using the commercial PCR purification kit (Qiagen, USA). Keeping the purified cDNA in 30.0 μl 1× TE buffer.

7. Hybridizing the cDNA with the mRNA Detection Probe; Amplifying the Hybridized cDNA; and Labeling the Amplified cDNA on the mRNA Microarray Chip

The specific detection portion of the mRNA detection probe on the mRNA microarray chip can hybridize the cDNA of the detecting mRNA.

The hybridized cDNA can be amplified through extending the polymer from the 3′end of the hybridized mRNA detection probe based on the ssDNA.

The amplified cDNA can be labeled through integrating the fluorescent modified nucleotide into the amplified duplex oligonucleotide during the polymer extension as shown in FIG. 3.

Applying the 10.0 μl 10× DNA polymerase reaction buffer, 30.0 μl cDNA solution, 10.0 μl 1 mM dNTPs (dATP, dCTP, dGTP), 7.5 μl 1 mM dTTP, 2.5 μl 1 mM dUTP-Cy5 (NEB, USA), 10 units DNA polymerase (NEB, USA), distill water to the final volume of 100.0 μl on the mRNA microarray chip.

Incubating the mRNA microarray chip at 50° C. for 8 hours.

Washing away the un-hybridized cDNA following the standard protocol.

8. Scanning the Hybridized and Amplified mRNA Microarray Chip

Scanning the hybridized and amplified mRNA microarray chip following the scanner's manufacture instructions (Affymetrix, USA).

9. Analyzing the Hybridized and Amplified Patterns of the Detecting mRNA

The fluorescent signal from the mRNA detection probe spot on the mRNA microarray chip will indicate the presence of the detected mRNA.

The quantity of the fluorescent signal from the mRNA detection probe spot on the mRNA microarray chip will be directly proportional to the amount of the integrated fluorescent modified nucleotide in the amplified duplex oligonucleotide, therefore, has close quantitative relationship with the detected mRNA.

What is the claim is:

1. The method of amplifying and labeling the message RNA (mRNA) sample for the mRNA microarray, comprising the following steps of:
a. extracting the mRNA from the cell or the tissue;
b. synthesizing the single strand specific DNA oligonucleotide (ssDNA);
c. applying the ssDNA, extracted mRNA, reverse transcriptase reaction buffer, reverse transcriptase and dNTPs mix into the tube to convert the mRNA to the complementary DNA (cDNA);
d. applying the RNAse in the tube to digest the mRNA from the polymer of cDNA/mRNA;
e. synthesizing the mRNA detection probe;
f. making the mRNA microarray chip through covalently attaching the spacer of the mRNA detection probe to the surface of the mRNA microarray chip;
g. applying the cDNA, DNA polymerase reaction buffer, DNA polymerase, dNTPs mix and fluorescent modified nucleotide onto the mRNA microarray chip to do the hybridization, amplification and labeling reaction;
h. scanning the hybridized, amplified and labeled mRNA microarray chip;
i. analyzing the hybridization pattern and amplification quantitation.

2. The method of claim 1, wherein the ssDNA is composed of two distinct portions, namely the ssDNA poly-T and the ssDNA tag. At the 3′end, the ssDNA poly-T is poly-deoxythymine oligonucleotide, while at the 5′end the universal sequence oligonucleotide functions as the ssDNA tag.

3. The method of claim 1, wherein the length of the ssDNA poly-T can be 6-30 nucleotides.

4. The method of claim 1, wherein the ssDNA poly-T can hybridize the complementary poly-adenosine (poly-A) in the tail of the mRNA.

5. The method of claim 1, wherein the cDNA can be extended from the 3′end of the hybridized ssDNA poly-T based on the hybridized mRNA to form the polymer of the cDNA/mRNA.

6. The method of claim 1, wherein the length of the ssDNA tag can be 50-200 nucleotides or longer.

7. The method of claim 1, wherein the ssDNA tag cannot contain any complementary nucleic acid sequence of the detecting mRNA.

8. The method of claim 1, wherein the ssDNA can be DNA, or RNA, or nucleic acid analogs.

9. The method of claim 1, wherein the mRNA detection probe on the mRNA microarray chip is composed of two distinct portions, namely the specific detection portion at the 3′end and the spacer at the 5′end.

10. The method of claim 1, wherein the mRNA detection probe on the mRNA microarray chip can be DNA or RNA or nucleonic acid analog.

11. The method of claim 1, wherein the whole length of the mRNA detection probe on the mRNA microarray chip can be 30-100 nucleotides or longer.

12. The method of claim 1, wherein the specific detection portion in the mRNA detection probe contains the sense sequence of the 3′end of the detecting mRNA with the sense sequence of the stop code at the 3′terminal of the mRNA detection probe.

13. The method of claim 1, wherein the specific detection portion of the mRNA detection probe on the microarray chip can hybridize the cDNA of the detecting mRNA.

14. The method of claim 1, wherein the DNA polymer can be extended from the 3′end of the hybridized mRNA detection probe on the mRNA microarray chip based on the ssDNA.

15. The method of claim 1, wherein the fluorescent modified nucleotide can be integrated into the amplified duplex oligonucleotide during the polymer extension.

16. The method of claim 1, wherein the fluorescent modified nucleotides can be dATP, or dCTP, or dGTP, or dTTP or dUTP.

17. The method of claim 1, wherein the fluorescent in the modified nucleotide can be any fluorescent dye that can be detected by the microarray chip scanner.

18. The method of claim 1, wherein the length of the spacer in the mRNA detection probe can be 8-10 nucleotides.

19. The method of claim 1, wherein the spacer in the mRNA detection probe cannot contain any complementary nucleic acid sequence of the detecting mRNA.

20. The method of claim 1, wherein the 5′terminal of the spacer of the mRNA detection probe is covalently attached to the surface of the mRNA microarray chip.
